(19) United States
(12) Patent Application Publication
(10) Pub. No.: US 2022/0323338 A1
(43) Pub. Date: Oct. 13, 2022

(54) MULTIFUNCTIONAL NATURAL FALSE EYELASH GLUE AND PREPARATION METHOD THEREOF

(71) Applicant: Qingdao Homay Eyelashes Co.,Ltd., Qingdao (CN)

(72) Inventor: Mingtao Zhang, Pingdu (CN)

(21) Appl. No.: 17/241,451

(22) Filed: Apr. 27, 2021

(30) Foreign Application Priority Data
Apr. 8, 2021 (CN) 202110376905.9

(51) Int. Cl. A61K 8/92; A61K 8/19; A61K 8/46; A61K 8/27; A61K 8/34; A61K 8/81; A61Q 1/10

(52) U.S. Cl. CPC A61K 8/922; A61K 8/19; A61K 8/46; A61K 8/27; A61K 8/345; A61K 8/8152; A61K 8/34; A61Q 1/10

(57) ABSTRACT

This disclosure relates to the field of cosmetics, and more particularly relates to a multifunctional natural false eyelash glue and a preparation method thereof. A multifunctional natural false eyelash glue, including, in parts by mass: 55˜65 parts of natural latex; 35˜40 parts of water; 0.1 part of ammonia; 0.4˜0.6 parts of potassium hydroxide; 0.4˜0.6 parts of tetramethylthiuram disulfide; 0.4 to 0.6 parts of zinc oxide; and 1 to 8 parts of ethylene glycol. A method of preparing a multifunctional natural false eyelash glue, comprising the following operations: (1) adding in sequence a natural latex, water, ammonia, potassium hydroxide, tetramethylthiuram disulfide, zinc oxide, and ethylene glycol to a container in amounts according to a formula, and mix them evenly to obtain the multifunctional natural false eyelash glue.

### CROSS-REFERENCE TO RELATED APPLICATIONS

This is a U. S. patent application which claims the priority and benefit of Chinese Patent Application Number 202110376905.9, filed on Apr. 8, 2021, the disclosure of which is incorporated herein by reference in its entirety.

### TECHNICAL FIELD

This disclosure relates to the field of cosmetics, and more particularly relates to a multifunctional natural false eyelash glue and a preparation method thereof.

### BACKGROUND

A false eyelash glue is mainly used to glue false eyelashes, artificial eyelashes for beautifying the eyes, so as to lengthen and thicken eyelashes. False eyelash glue is not only used in professional makeup, but also used in daily makeup.

Currently, most of the false eyelash glues on the market are chemical blended products. Chemically blended false eyelash glues have defects such as ease of triggering allergies, difficulty to clean, and excessive hazardous substances and heavy metals. The very few remaining natural rubber-based false eyelash glues also have serious defects: they are difficult to clean, don't like low temperatures, have insufficient adhesion, and are added with various fragrance, and so lose their pure natural property.

### SUMMARY

It is therefore an object of this disclosure to solve the above-mentioned problems of false eyelash glues which are difficult to clean, intolerant of low temperature, have insufficient adhesion, and have excessive addition of fragrance, and accordingly discloses a multifunctional natural false eyelash glue and a preparation method thereof.

Disclosed is the following technical solution: A multifunctional natural false eyelash glue, including, in parts by mass:

55˜65 parts of natural latex;

35˜40 parts of water;

0.1 part of ammonia;

0.4˜0.6 parts of potassium hydroxide;

0.4˜0.6 parts of tetramethylthiuram disulfide;

0.4˜0.6 parts of zinc oxide; and

1˜8 parts of ethylene glycol.

Further, it includes 1˜6 parts of carbon black.

Furthermore, it includes 30˜50 parts of a blending liquid, which includes:

2˜3% of propylene glycol;

30˜50% of iron oxide;

20˜35% of styrene/acrylic (ester) copolymer;

0.08˜0.09% of ethylhexylglycerin;

0.23˜0.24% of caprylyl glycol;

0.26˜0.27% of phenoxyethanol;

and the rest is water.

There is provided a method for preparing a multifunctional natural false eyelash glue, which includes the following operations:

(1) Add in sequence a natural latex, water, ammonia, potassium hydroxide, tetramethylthiuram disulfide, zinc oxide, and ethylene glycol to a container in amounts according to a formula, and mix them evenly to obtain a multifunctional natural false eyelash glue.

Further, the method includes operation (2):

Add an amount of carbon black according to the formula to the above-mentioned multifunctional natural false eyelash glue, and mix it evenly.

Further, it includes operation (3) of adding a blending liquid, including:

(31) Prepare the blending liquid:

Add appropriate amounts of water, propylene glycol, iron oxide, styrene/acrylic (ester) copolymer, ethylhexylglycerin, caprylyl glycol, phenoxyethanol to the container according to the formula amounts of the blending liquid. After stirring for 60 minutes under 20-30° C., the blended solution will be obtained after standing for 8 hours;

(32) Add the blending liquid in a formula amount to the multifunctional natural false eyelash glue, then stir under 20˜′30° C. for 20˜60 minutes, filter with a pressure filter, and then obtain the multifunctional natural false eyelash glue after filtration.

Further, the obtained multifunctional natural false eyelash glue is filtered with a pressure filter with a mesh pore size of not less than 800 mesh.

The multifunctional natural false eyelash glue and its preparation method disclosed in the present disclosure have the following beneficial effects.

1. This product uses pure natural latex, is prepared with fine processing, anti-allergic, has good adhesion, contains no harmful substances, and can absorb part of the water on the human skin when sweating thus keeping the skin pores unobstructed.

2. It is very easy to clean up. Whether it is applied to the skin or on the eyelash stalks, gently rub or pinch a corner and gently tear it off in strips. There is no need to worry about tearing the false eyelashes when cleaning up due to glue problems. It can also be used as a disposable eyelash grafting glue.

3. The glue of this disclosure overcomes the shortcomings of all previous natural latex false eyelash glues that are not resistant to low temperatures, cannot be shipped normally below zero, cannot be used normally, and cannot be reused after freezing. This product can still be used normally at minus 18° C., even if the temperature is even lower and freezes, it can still be used normally after melting.

4. It contains iron oxide, which attracts magnets and magnetic false eyelashes, which is convenient to wear and easy to clean up, by just rubbing or tearing it off gently. When the magnet of the magnetic false eyelashes falls off, the glue can also be used as a regular natural glue.

### DETAILED DESCRIPTION

Specific embodiments of the present disclosure will be described in detail below.

### Specific Embodiment 1

A multifunctional natural false eyelash glue, including, in parts by mass:

55 parts of natural latex;

35 parts of water;

0.1 part of ammonia;

0.4 parts of potassium hydroxide;

0.4 parts of tetramethylthiuram disulfide;

0.4 parts of zinc oxide; and

1 part of ethylene glycol.

Further, it includes 1 part of carbon black.

Furthermore, it includes 30 parts of a blending liquid, which includes:

2% of propylene glycol;

30% of iron oxide;

20% of styrene/acrylic (ester) copolymer;

0.08% of ethylhexylglycerin;

0.23% of caprylyl glycol;

0.26% of phenoxyethanol;

and the rest is water.

There is provided a method for preparing a multifunctional natural false eyelash glue, which includes the following operations:

Add in sequence a natural latex, water, ammonia, potassium hydroxide, tetramethylthiuram disulfide, zinc oxide, and ethylene glycol to a container in amounts according to a formula, and mix them evenly to obtain a multifunctional natural false eyelash glue.

Further, the method includes operation (2):

Add an amount of carbon black according to the formula to the above-mentioned multifunctional natural false eyelash glue, and mix it evenly.

Further, it includes operation (3) of adding a blending liquid, including:

(31) Prepare the blending liquid:

Add appropriate amounts of water, propylene glycol, iron oxide, styrene/acrylic (ester) copolymer, ethylhexylglycerin, caprylyl glycol, phenoxyethanol to the container according to the formula amounts of the blending liquid. After stirring for 60 minutes under 30° C., the blended solution will be obtained after standing for 8 hours;

(32) Add the blending liquid in a formula amount to the multifunctional natural false eyelash glue, then stir under 30° C. for 60 minutes, filter with a pressure filter, and then obtain the multifunctional natural false eyelash glue after filtration.

Further, the obtained multifunctional natural false eyelash glue is filtered with a pressure filter with a mesh pore size of not less than 800 mesh.

### Specific Embodiment 2

A multifunctional natural false eyelash glue, including, in parts by mass:

65 parts of natural latex;

40 parts of water;

0.1 part of ammonia;

0.6 parts of potassium hydroxide;

0.6 parts of tetramethylthiuram disulfide;

0.6 parts of zinc oxide; and

8 part of ethylene glycol.

Further, it includes 6 part of carbon black.

Furthermore, it includes 50 parts of a blending liquid, which includes:

3% of propylene glycol;

50% of iron oxide;

35% of styrene/acrylic (ester) copolymer;

0.09% of ethylhexylglycerin;

0.24% of caprylyl glycol;

0.27% of phenoxyethanol;

and the rest is water.

There is provided a method for preparing a multifunctional natural false eyelash glue, which includes the following operations:

Add in sequence a natural latex, water, ammonia, potassium hydroxide, tetramethylthiuram disulfide, zinc oxide, and ethylene glycol to a container in amounts according to a formula, and mix them evenly to obtain a multifunctional natural false eyelash glue.

Further, the method includes operation (2):

Add an amount of carbon black according to the formula to the above-mentioned multifunctional natural false eyelash glue, and mix it evenly.

Further, it includes operation (3) of adding a blending liquid, including:

(31) Prepare the blending liquid:

Add appropriate amounts of water, propylene glycol, iron oxide, styrene/acrylic (ester) copolymer, ethylhexylglycerin, caprylyl glycol, phenoxyethanol to the container according to the formula amounts of the blending liquid. After stirring for 60 minutes under 30° C., the blended solution will be obtained after standing for 8 hours;

(32) Add the blending liquid in a formula amount to the multifunctional natural false eyelash glue, then stir under 30° C. for 20 minutes, filter with a pressure filter, and then obtain the multifunctional natural false eyelash glue after filtration.

Further, the obtained multifunctional natural false eyelash glue is filtered with a pressure filter with a mesh pore size of not less than 800 mesh.

### Specific Embodiment 3

A multifunctional natural false eyelash glue, including, in parts by mass:

60 parts of natural latex;

38 parts of water;

0.1 part of ammonia;

0.5 parts of potassium hydroxide;

0.5 parts of tetramethylthiuram disulfide;

0.5 parts of zinc oxide; and

4 part of ethylene glycol.

Further, it includes 3 part of carbon black.

Furthermore, it includes 40 parts of a blending liquid, which includes:

2.5% of propylene glycol;

40% of iron oxide;

25% of styrene/acrylic (ester) copolymer;

0.085% of ethylhexylglycerin;

0.235% of caprylyl glycol;

0.265% of phenoxyethanol;

and the rest is water.

There is provided a method for preparing a multifunctional natural false eyelash glue, which includes the following operations:

Add in sequence a natural latex, water, ammonia, potassium hydroxide, tetramethylthiuram disulfide, zinc oxide, and ethylene glycol to a container in amounts according to a formula, and mix them evenly to obtain a multifunctional natural false eyelash glue.

Further, the method includes operation (2):

Add an amount of carbon black according to the formula to the above-mentioned multifunctional natural false eyelash glue, and mix it evenly.

Further, it includes operation (3) of adding a blending liquid, including:

(31) Prepare the blending liquid:

Add appropriate amounts of water, propylene glycol, iron oxide, styrene/acrylic (ester) copolymer, ethylhexylglycerin, caprylyl glycol, phenoxyethanol to the container according to the formula amounts of the blending liquid. After stirring for 60 minutes under 25° C., the blended solution will be obtained after standing for 8 hours;

(32) Add the blending liquid in a formula amount to the multifunctional natural false eyelash glue, then stir under 25° C. for 40 minutes, filter with a pressure filter, and then obtain the multifunctional natural false eyelash glue after filtration.

Further, the obtained multifunctional natural false eyelash glue is filtered with a pressure filter with a mesh pore size of not less than 800 mesh.

The embodiments of the present disclosure have been described in detail above. However, the present disclosure will not be limited to the above-mentioned embodiments, and various changes can be made without departing from the spirit and scope of the present disclosure within the scope of knowledge possessed by those having ordinary skill in the art.

What is claimed is:

1. A multifunctional natural false eyelash glue, comprising, in parts by mass:
55˜65 parts of natural latex;
35˜40 parts of water;
0.1 part of ammonia;
0.4˜0.6 parts of potassium hydroxide;
0.4˜0.6 parts of tetramethylthiuram disulfide;
0.4˜0.6 parts of zinc oxide; and
1˜8 parts of ethylene glycol.

2. The multifunctional natural false eyelash glue of claim 1, further comprising 1 to 6 parts of carbon black.

3. The multifunctional natural false eyelash glue of claim 2, further comprising 30˜50 parts of a blending liquid, the blending liquid comprising:
2˜3% of propylene glycol;
30˜50% of iron oxide;
20˜35% of styrene/acrylic (ester) copolymer;
0.08˜0.09% of ethylhexylglycerin;
0.23˜0.24% of caprylyl glycol;
0.26˜0.27% of phenoxyethanol; and
water.

4. A method for preparing a multifunctional natural false eyelash glue, comprising:
(1) adding in sequence a natural latex, water, ammonia, potassium hydroxide, tetramethylthiuram disulfide, zinc oxide, and ethylene glycol to a container in amounts according to a formula, and mix them evenly to obtain the multifunctional natural false eyelash glue.

5. The method of claim 4, further comprising operation (2):
adding an amount of carbon black according to the formula to the above-mentioned multifunctional natural false eyelash glue, and mixing it evenly.

6. The method of claim 5, further comprising operation (3) of adding a blending liquid, comprising:
(31) preparing the blending liquid:
adding appropriate amounts of water, propylene glycol, iron oxide, styrene/acrylic (ester) copolymer, ethylhexylglycerin, caprylyl glycol, phenoxyethanol to the container according to the formula amounts of the blending liquid, stirring for 60 minutes under 20-30° C., and standing them for 8 hours to obtain the blending liquid;
(32) adding the blending liquid in an amount according to the formula to the multifunctional natural false eyelash glue, stirring under 20˜30° C. for 20˜60 minutes, filtering with a pressure filter, and obtaining the multifunctional natural false eyelash glue after filtration.
